(19) United States
(12) Patent Application Publication
(10) Pub. No.: US 2011/0112511 A1
(43) Pub. Date: May 12, 2011

(54) METHOD AND APPARATUS FOR ADMINISTERING ANESTHETICS TO PERIPHERAL NERVE REGIONS

(76) Inventor: Jonathan E. Singer, Port Washington, NY (US)

(21) Appl. No.: 12/614,743

(22) Filed: Nov. 9, 2009

(51) Int. Cl. A61M 19/00; A61M 25/06

(52) U.S. Cl. 604/512; 604/158

(57) ABSTRACT

A method and apparatus for administering liquid anesthetics around peripheral nerves, in order to perform surgery or to relieve post operative pain. The apparatus containing a regional anesthetic needle with an overlying catheter. The needle is hollow and has a blunt end so that it does not penetrate or damage a nerve or blood vessel. A stiff catheter covers the needle up to a side hole in the needle that allows liquid anesthetic to be injected through the needle in a forward direction. The needle can be removed leaving catheter in place without disturbing the location of the catheter.

### BACKGROUND OF THE INVENTION

1. Field of the Invention

This invention relates to pain management systems, and more specifically, to a catheter-based infusion system for the administration of anesthetic fluids to a patient. In particular, the invention relates to a novel method and apparatus for performing a continuous peripheral nerve block procedure on a human patient.

2. State of the Art

Currently in the medical field, continuous interscalene blocks are utilized for intraoperative and postoperative pain management for patients in major surgery of the upper and lower extremities. In regard to the upper extremities, it can be used, for example, in shoulder arthroplasty or rotator cuff repair. It can also be used for surgery or post operative pain for arm and hand surgery. Additionally, it can be used in any kind of surgery on the lower extremities, such as arthroscopy of the knee with graft repair, total knee replacement, total ankle replacement, arthroscopy of the ankle, or podiatry surgery.

At present with the currently employed ultrasound technique, there is only one way of placing a continuous regional anesthetic catheter in a patient which is by passing a catheter through an epidural-like needle such as that described in U.S. Pat. No. 5,843,048 to Gross (which is incorporated herein by reference).

In general, this procedure is performed by inserting a hollow, echogenic (visible by ultrasound) epidural-like “introducer” needle into the patient's skin close to the nerve bundle to be anesthetized, advancing a mostly non-echogenic (invisible to ultrasound) catheter through the needle and then verifying placement of the catheter by ultrasound imaging, after injecting an echogenic translucent liquid anesthetic, through the catheter. With the current technique, the “introducer” needle must then be removed from over the catheter, which often disadvantageously leads to dislodgement or misplacement of the location of the catheter.

More particularly, according to the currently employed procedure, a sterile ultrasound transducer probe is held in one hand by the doctor or individual performing the procedure. A sterile, epidural-like needle is held in the other hand and is advanced to pierce the patient's skin in the desired area. The needle is then advanced towards the nerve bundle to be anesthetized. Proper placement of the position of the needle is assured by visualization of its position under the patient's skin with the aid of ultrasound imaging.

Particularly, an echogenic liquid anesthetic fluid can be injected through the needle, while observing the area with ultrasound imaging. The translucent property of the liquid anesthetic fluid aids in identifying the position of the tip of the needle relative to the nerve bundle to be anesthetized, since it is translucent as opposed to the other structures and, thus, visible via ultrasound imaging. The method also requires visualizing the nerve bundle with the ultrasound imaging and injecting the echogenic fluid around the nerve to assure that the desired nerves are being bathed by the fluid.

Once proper needle placement has been assured by visualizing the position of the tip of the needle ultrasonically, the ultrasound probe is removed so that the doctor has two free hands. Alternatively, the ultrasound probe can be utilized if a second individual or device can hold the ultrasound transducer probe under sterile conditions. Next, the needle is left in place and a catheter, that cumbersomely requires both hands for placement, is then threaded through the center of the needle. It is typically difficult to thread the catheter through the needle and may require that the needle be rotated to facilitate advancement of the catheter. This can result in the physician having no control over the direction of the catheter once it is advanced past the tip of the needle.

A syringe attachment device is then connected to the distal end of the catheter. With one hand on the catheter, holding the syringe that has been attached to it and the other hand holding the ultrasound probe, the ultrasound is then used once again to identify the nerves to be anesthetized. This time the catheter with attached anesthetic syringe, rather than the needle, is injected with echogenic liquid anesthetic to visualize, with the aid of the ultrasound probe, the location of the catheter tip. The catheters are currently non-echogenic which means essentially that they cannot be seen with ultrasound imaging unless the tip is visualized in a head-on direction.

Alternatively, air can be injected through the catheter to determine the location of the tip of the catheter. As another alternative, instead of using liquid anesthetic, the location of the catheter can be determined by the use of a stimulating needle. The stimulating needle is used to approximate the location of the nerve bundle by using electrical stimulation and observing the muscle response. Furthermore, a stimulating catheter can be utilized to determine whether the catheter has been displaced.

The typically flexible, non-rigid, thin catheter can curve or curl as it is advanced through the needle and comes into contact with the patient's tissue. There is no way to ensure the direction of the catheter once it is advanced past the tip of the needle, since it will often be directed towards the patient's tissue with the least resistance. Consequently, often the tip of the catheter is not located properly and needs to be repositioned to an accurate position. Once again, in an unduly time consuming manner, the location of the tip is verified by injecting the translucent liquid anesthetic. If the catheter has curved, the fluid will inaccurately end up either too far below or past the nerve and will be ineffective. If the catheter is not properly located after the visual method described, then undesirably, the entire process has to be repeated including the step of pulling out the main needle with the catheter in it, to avoid shearing the catheter off in the patients' tissue.

Once the desired placement of the catheter is verified by visualization with ultrasound imaging to ensure that the catheter is located adjacent to the nerve bundle, the next step involves removing the ultrasound transducer probe, removing the syringe attachment device from the catheter, and removing in a cumbersome fashion the epidural-like needle which overlies the catheter, while leaving the catheter in place. Presently, the only way to hold the catheter in place is to push it forward while removing the needle, until the needle is completely removed from the patient. The physician must then grab the end of the catheter before the needle is removed from the catheter. Thus, removal of the overlying needle often disadvantageously results in misplacement, and sometimes complete dislodgement of the catheter, as there is no way to assure that the catheter remains still as the needle is removed. This is particularly true in obese people, where the physician has to use a longer needle, for example, four (4″) to five (5″) inch needles.

To assure proper placement of the catheter, the syringe attachment device must then be re-attached to the catheter and the desired location of the catheter must again be verified by injection and visualization of the tip of the catheter via the ultrasound. It is also important to note again, that because the catheter is not echogenic, it is difficult to visualize its location with the needle removed. Thus, it may require multiple injections of liquid anesthetic to locate the catheter tip with ultrasound which is unduly time-consuming.

Therefore, when the needle is being pulled out over the catheter, the prior conventional method can undesirably cause the catheter to advance, or at a minimum, exert pressure inward on it to avoid it coming out. Thus, the ultrasound may have to be used multiple times to relocate the catheter relative to the nerve bundle.

Furthermore, these catheters can unsafely be positioned in large blood vessels or in the nerve bundle itself. If the catheter is placed into the blood vessel and a large dose of anesthetic is administered, the results could be catastrophic and the patient can have a cardiac arrest due to local anesthetic toxicity and die, once the liquid anesthetic is injected. If the catheter is placed in the nerve bundle, it can undesirably cause permanent nerve damage. Furthermore, if the catheter unknowingly remains in a blood vessel the anesthetic may be pumped into the patient's blood stream for days, which could be catastrophic.

The prior art discloses catheter-based infusion systems and methods of administering anesthetic fluid to a patient. However, so far as is known, none of the prior art devices or methods addresses all of the above-noted problems in a simple, effective and yet highly advantageous manner as does the present invention discussed herein.

### SUMMARY OF THE INVENTION

It is therefore an object of the invention to provide a novel catheter-based infusion system and method for the administration of anesthetic fluid to a patient.

It is also an object of the invention to provide a method and an apparatus which will aid in the more efficient and accurate placement of a catheter-based infusion system for the administration of anesthetic fluid to a patient.

It is an object of the invention to also provide a method and apparatus which minimizes dislodgement or shifting of the location of the catheter and the need to use ultrasound to verify placement of the tip of the catheter.

It is another object of the invention to provide an apparatus and method for safer placement of a catheter-based infusion system.

It is yet another object of the invention to provide an apparatus and method to provide a catheter-based infusion system which is less cumbersome and time-consuming to position within a patient.

It is a further object of the invention to provide an apparatus and method for placement of a catheter-based infusion system intended to minimize catastrophic consequences.

It is another object of the invention to provide an apparatus and method for placement of a catheter-based infusion system which is easy to produce and is more cost effective.

It is yet another object of the invention to provide an apparatus and method for placement of a catheter-based infusion system which can be performed by a single individual.

Certain of the foregoing and related objects are readily attained according to the present invention by the provision of an assembly for continuous administration of regional liquid anesthetic, comprising a needle having an open proximal end, a distal end opposite therefrom, an opening generally adjacent to said distal end, and a lumen extending through said needle such that said open proximal end of said needle is in fluid communication with said opening. The assembly further comprises a catheter having an open proximal end, an open distal end opposite therefrom and a lumen extending through said catheter such that said proximal end of said catheter is in fluid communication with said distal end of said catheter. The needle, in a first position, is removably mounted telescopically within said catheter and is arranged to extend through said first fluid port hub and said lumen of said catheter, so that said catheter fits over said needle. The opening of said needle is located outwardly and distally of said open distal end of said catheter and in which position said needle and catheter distal ends may be advanced in tandem to a desired position within the patient. In a second position, said needle is slidably withdrawn and slidably removed from said catheter upon hand manipulation without altering the location of said catheter within the patient.

Preferably, said opening is located on a side of said needle and said distal end of said needle is closed. Desirably, the distal end of said needle is blunt and in a further preferred embodiment, the catheter is relatively rigid. Advantageously, said needle is removable from said catheter by holding said catheter and pulling on said needle.

In yet another preferred embodiment, said assembly further comprises, a first fluid port hub coupled to said proximal end of said catheter and in fluid communication with said lumen of said catheter; and, a second fluid port hub coupled to said proximal end of said needle and in fluid communication with said lumen of said needle wherein said first and second fluid port hubs are adapted to be coupled to a source of liquid anesthetic. Desirably, said first and second fluid port hubs are configured and dimensioned to accept a medical syringe. In a preferred embodiment, said second fluid port hub is configured and dimensioned for receipt within said first fluid port hub in a friction-fit manner.

Advantageously, said distal end of said catheter is located generally adjacent to said open proximal end of said needle and said open proximal end of said needle is angled distally. Also in a preferred embodiment, said needle is a stimulating needle and said catheter is a stimulating catheter.

Certain of the foregoing and related objects are readily attained according to the present invention also by the provision of a method of placing a catheter for continuous administration of a peripheral regional anesthetic in a patient, said method comprising the steps of providing a needle having an open proximal end, a distal end opposite therefrom, an opening generally adjacent to said distal end, and a lumen extending through said needle such that said open proximal end of said needle is in fluid communication with said opening and a catheter having an open proximal end, an open distal end opposite therefrom, and a lumen extending through said catheter such that said proximal end of said catheter is in fluid communication with said distal end of said catheter. In a first position, said needle is removably mounted telescopically within said catheter and is arranged to extend through said first fluid port hub and said lumen of said catheter, so that said catheter fits over said needle, and with said opening of said needle located outwardly and distally of said open distal end of said catheter and in which position said needle and catheter distal ends may be advanced in tandem to a desired position with the patient. In a second position, said needle is withdrawn and slidably removed from said catheter upon hand manipulation without altering the location of said catheter within the patient. The next step is inserting said needle and catheter assembly into a patient, towards a region to be anesthetized. Next, is locating said distal opening of said needle with the aid of imaging and removing said needle from inside said catheter while holding said catheter in place in a patient.

Preferably, the method further comprises injecting liquid anesthetic through said needle while imaging. In another preferred embodiment, the step comprises providing a first fluid port hub coupled to said proximal end of said catheter and in fluid communication with said lumen of said catheter; and a second fluid port hub coupled to said proximal end of said needle and in fluid communication with said lumen of said needle and coupling a source of liquid anesthetic to said proximal hub of said catheter, after said needle is removed. Preferably, the location of said catheter is secured with a topical dressing.

In yet another preferred embodiment, the step of imaging is performed with ultrasound and the step of imaging includes injecting an echogenic liquid into said needle.

Advantageously, said needle is a stimulating needle and said catheter is a stimulating catheter.

Other objects and features of the present invention will become apparent from the detailed description considered in connection with the accompanying drawings, which disclose several embodiments of the invention. It is to be understood that the drawings are to be used for the purpose of illustration only and not as a definition of the limits of the invention.

### BRIEF DESCRIPTION OF THE DRAWINGS

FIG. 1 is a perspective view of a catheter-based infusion system for the administration of anesthetic fluid to a patient, according to the present invention;

FIG. 2 is a longitudinal sectional view of the catheter-based infusion system taken along line 2-2 in FIG. 1 with the needle rotated 90°;

FIG. 3 is an enlarged broken sectional view showing the distal ends of the catheter and the needle of the catheter-based infusion system shown in FIG. 2;

FIG. 4 is an enlarged, broken sectional view similar to FIG. 3 of the catheter-based infusion system but rotated 90° about the coaxial longitudinal axes of the catheter and needle;

FIGS. 5-11 are a series of pictorial sketches illustrating the method steps of administering anesthetic fluids by the catheter-based infusion system according to the invention.

### DETAILED DESCRIPTION OF THE PRESENTLY PREFERRED EMBODIMENTS

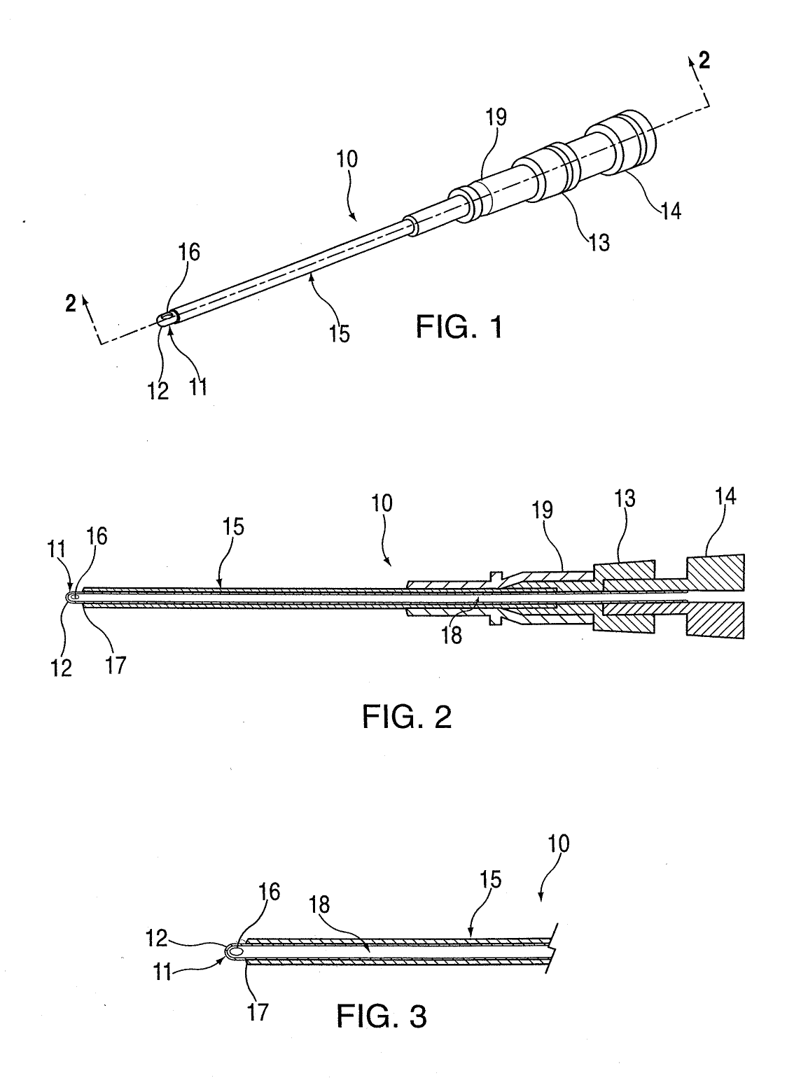

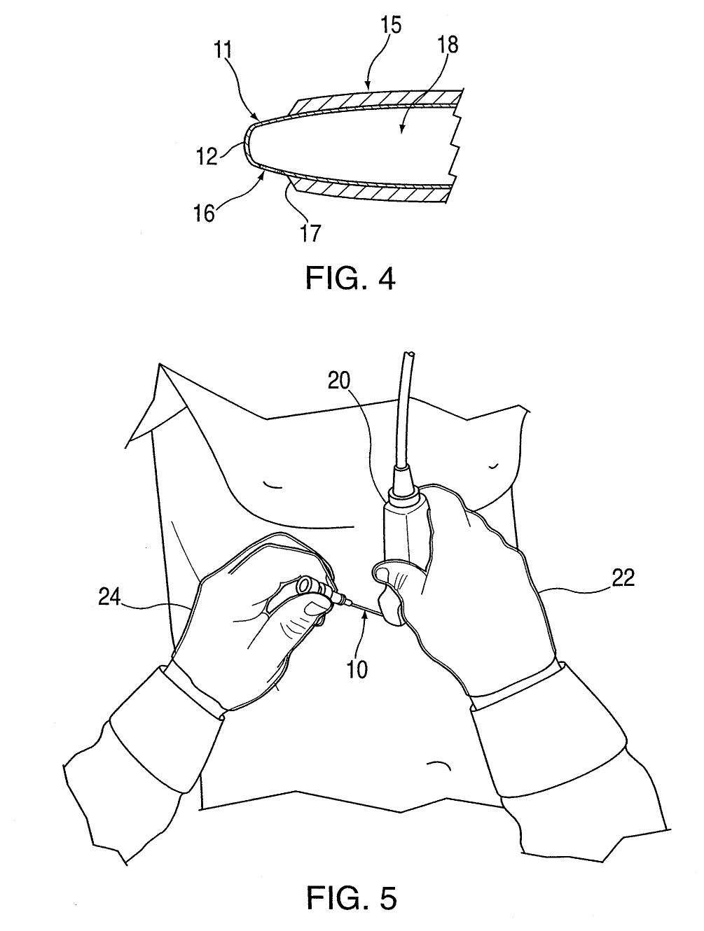

Turning now in detail to the drawings, FIGS. 1-4 thereof illustrate a novel catheter-based assembly 10 for the administration of anesthetic fluid to a patient, according to the invention. As shown in FIGS. 1 and 2, the assembly 10 includes a needle 11 which is in the form of a standard, hollow hypodermic needle for insertion into a patient. The needle has a forward or distal end or tip 12 for insertion into a patient and a rearward or proximal end, which is coupled to a needle hub or fluid port hub 14, so that it can be coupled to a source of fluid, such as a liquid anesthetic fluid. More particularly, the fluid port hub 14 is configured and dimensioned so that it is capable of accepting a standard medical syringe to allow for injection of fluid into the patient's body, e.g., via a conventional Luer connection such as that illustrated and described in U.S. Pat. No. 5,843,048 to Gross (which is incorporated herein by reference). However, it can be appreciated that other sources of fluid can be utilized.

The needle can range in length, for example, a one inch (1″), two inch (2″), three inch (3″) or four inch (4″) or half inch length needles and needles of different gauges can be used, such as 18, 19, 20, 21 or 22 gauge. Accordingly, it can be appreciated that the length and gauges of the needle utilized can vary depending on the procedure and patient. Furthermore, the needle 11 can be echogenic which allows it to be visualized with ultrasound imaging but the tip may still be difficult to visualize. Alternatively, the needle 11 can be a stimulating needle which can be attached to a conventional nerve stimulator in order to locate the nerves.

As best illustrated in FIGS. 1-4, according to the invention, the distal end 12 of the needle 11 is not pointed but constructed and dimensioned purposely blunt. The blunt distal end 12 is such that a person having ordinary skill in the art would appreciate that it is sharp enough to penetrate skin, muscle, fascia, adipose tissue, and other soft tissues of the body but which is not sharp enough to penetrate a blood vessel or a nerve bundle, while using the force generally used when performing the present procedure. For example, a needle as set forth in U.S. Pat. No. 5,843,048 to Gross (which is incorporated herein by reference) is sufficiently blunt to be utilized. However, U.S. Pat. No. 4,828,549 to Kvalo (which is incorporated herewith by reference) discloses a needle which is too sharp to be utilized with the present invention, as it is intended to pierce and to introduce a catheter into a patient's blood vessel.

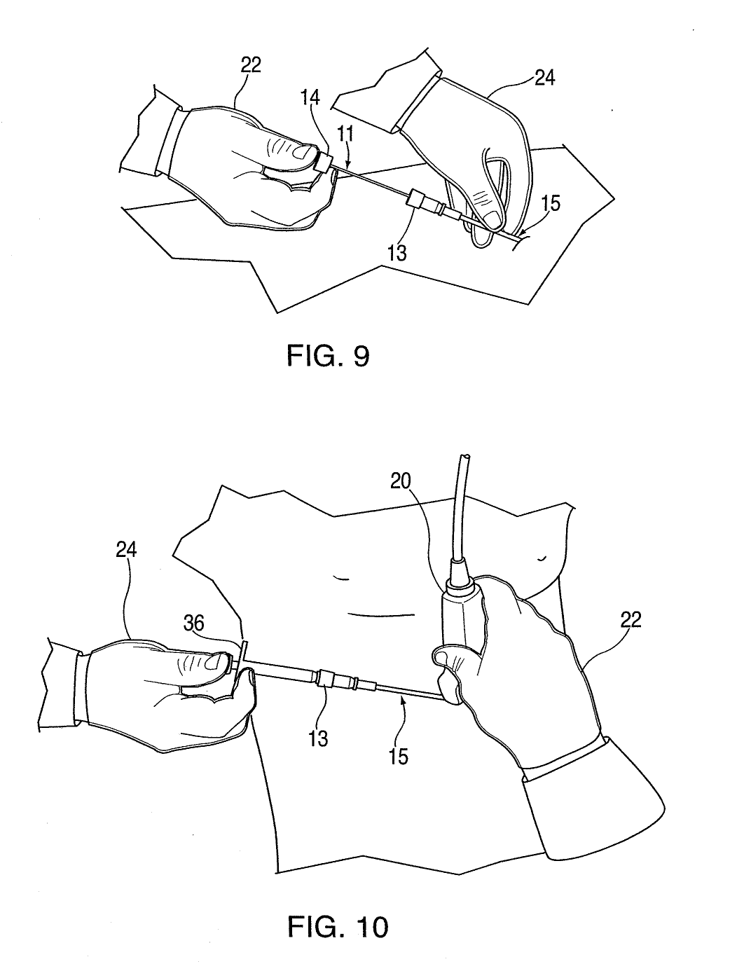

As seen in FIGS. 1-3, the needle 11 has a side opening or hole 16 which is located generally adjacent to the distal end 12 of the needle. As illustrated in FIG. 2, a lumen or channel 18 is formed within the needle 11 which extends from the fluid port hub 14 to the side opening 16, and through which a fluid can flow. Particularly, the fluid port hub 14 is adapted to be coupled to a source of fluid, in particular, a source of liquid anesthetic fluid. More particularly, it is configured and dimensioned to accept a medical syringe 36 e.g., via the conventional Luer connection. This allows for insertion of a liquid anesthetic into the needle 11 which then flows through the lumen 18 and out of the needle 11 via the side opening 16. This allows for visualization of the tip of the needle 11 by ultrasound imaging due to the transparency of the liquid anesthetic fluid relative to the other structures visualized. As seen best in FIG. 4, the side opening 16 of the needle 11 is angled forward distally so that fluid which exits the needle is directed in the forward distal direction.

As seen in FIG. 2, the assembly 10 also includes a hollow catheter 15 which overlies and covers a portion of the needle 11. The catheter can be generally non-echogenic which means that it cannot be visualized by ultrasound imaging, unless it is visualized in a head-on direction. The catheter has a forward or distal end or tip 17 which is generally adjacent to the distal end 12 of the needle 11, and a proximal end that is attached to a catheter hub or fluid port hub 13 which is also capable of being coupled to a source of fluid. Particularly, the fluid port hub 13 is configured and dimensioned so that it is capable of accepting a medical syringe 36 or medical tubing, e.g., via a conventional Luer connection, such as that in U.S. Pat. No. 5,843,048 to Gross, to allow for the injection of fluid into a patient. However, it can be appreciated that other sources of fluid can be utilized. The hub 14 of the needle 11 is configured and dimensioned for receipt within the hub 13 of the catheter 15 when the needle 11 is within the catheter 15.

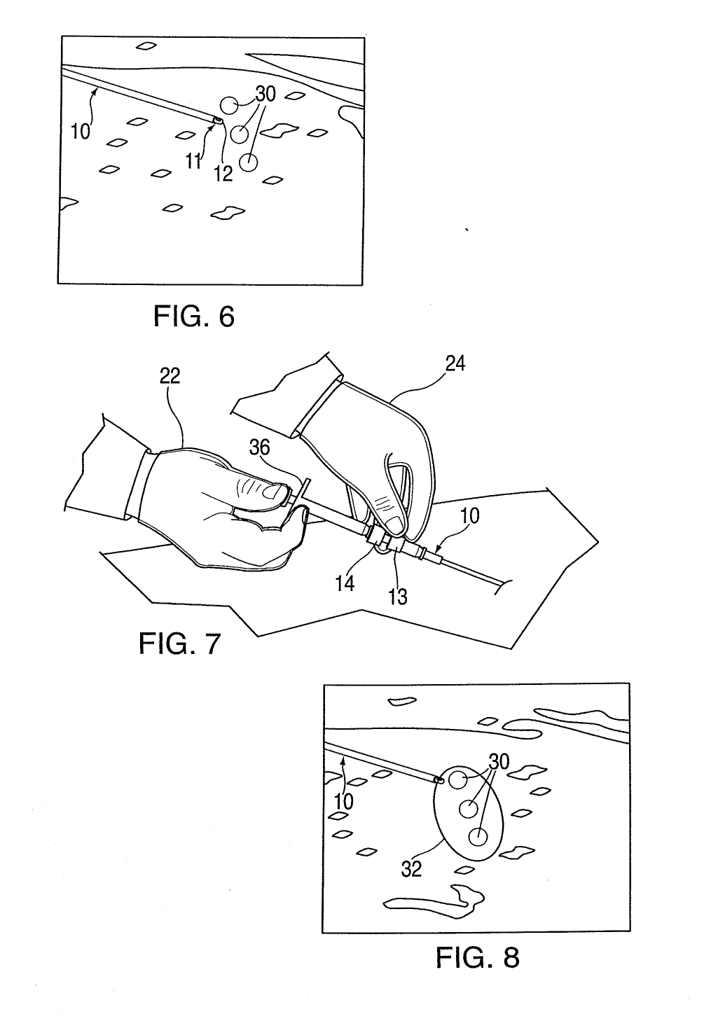

As seen in FIG. 3, the distal end 17 of the catheter 15 is open when the needle 11 is removed, to allow for fluid to exit the catheter. This allows for visualization of the distal end or tip 17 of the catheter by ultrasound imaging due to the transparency of the liquid anesthetic fluid relative to the other structures visualized. The catheters are typically non-echogenic and are made of a polyamide material that slides easily over metal. Other materials such as Teflon® or nylon can be used. It can be appreciated that various materials can be used in the catheter which allow for easy removal of the needle from the catheter. The catheter is thin and flexible but is sufficiently stiff or rigid in order to minimize the catheter from being bent or kinked which would occlude the lumen of the catheter. A catheter such as that in the CONTIPLEX® Tuohy Continuous Nerve Block Set REF 00331693, Product Code CNB400 manufactured by B. Braun Medical Inc. of Bethlehem, Pa. would be of suitable flexibility and rigidity to be utilized. However, the catheter 15 of the present invention has two open ends and not a closed tip, as in the CONTIPLEX® catheter. Alternatively, an echogenic catheter can be utilized in order to visualize the location of the tip of the catheter.

As best illustrated in FIG. 2, a needle 11 is received within a lumen or channel formed within the catheter 15, which extends the length of the catheter from the fluid port hub 13 to the open distal end 17. The catheter lumen (not visible) allows for fluid, i.e., a liquid anesthetic, which can be injected via a medical syringe 36 (FIG. 10) connected to the fluid port hub 13, to flow into the hub and out through the open distal end 17, when the needle is removed, in order to visualize the location of the tip of the catheter and to administer local anesthetic.

As seen in FIGS. 2-4, the catheter 15 fits over the needle 11 in a sheath or telescopic-like manner except for the tip 12 and opening 16. More particularly, the needle is configured and dimensioned to extend through the fluid port hub 13 of the catheter and extends through the lumen of the catheter 15 and extends somewhat beyond the catheter tip 17. The distal end 17 of the catheter 15 is provided proximally or rearward of the side opening 16 in the needle 11, so that the catheter does not overlie or cover the side opening 16 in the needle, to allow for fluid to exit the side opening 16 while the catheter overlies the needle. Furthermore, the distal end 17 of the catheter is provided generally adjacent to the side opening 16 of the needle 11, so that visualization of the tip of the needle approximates the tip or distal end 17 of the catheter.

The needle 11 serves the function of positioning or advancing the overlying catheter 15 into the tissues surrounding the nerve bundle to be anesthetized, as both the needle and catheter are advanced simultaneously in tandem to the desired area. The catheter 15 is configured and dimensioned such that it is removably secured over the needle 11 in a sheath-like manner having a snug fit, so that the position of the catheter 15 is not altered when the assembly 10 is advanced and positioned within a patient, but allows for removal of the needle without altering the placement of the catheter. For example, a catheter one gauge larger than the needle can be utilized, such as, a 20 gauge needle, with a 19 gauge catheter; a 19 gauge needle with an 18 gauge catheter; or a 18 gauge needle with a 17 gauge catheter. The diameter of the catheter lumen must be of a sufficient size so that it can accommodate and receive the needle in a telescopic manner.

More particularly, the position of the catheter and needle are fixed relative to one another, preferably with a friction fit, and are advanced together as a single unit since the hub 14 of the needle 11 is configured and dimensioned for receipt within the hub 13 of the catheter 15 to prevent movement of the catheter relative to the needle. As seen in FIGS. 1 and 2, the catheter 15 and hub 13 are connected together via a forward catheter housing 19. The suitable structure of such needle and catheter hubs which can be utilized is set forth in U.S. Pat. No. 4,828,549 to Kvalo. A notch can also be provided in the hub of the catheter into which a corresponding portion of the needle hub fits, in order to prevent rotation of the needle relative to the catheter (not shown).

The needle 11 is slidably removable and withdrawn from the catheter 15 by holding the catheter in place and pulling on the needle 11 by hand manipulation. As discussed above, the assembly 10 is configured and dimensioned such that the needle 11 can be easily removed from the catheter without dislodging or altering the location or placement of the catheter 15 within the patient.

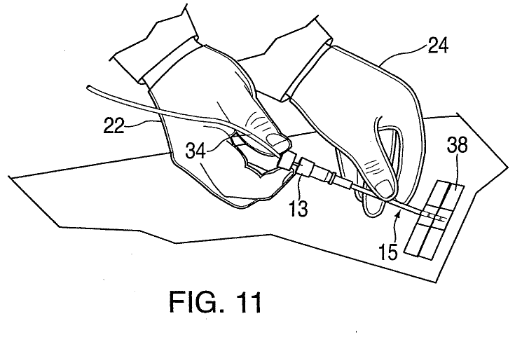

Turning now to FIGS. 5-11, therein illustrated are the steps of the method of administering anesthetic fluid to a patient according to the catheter-based infusion system of the invention. In particular, as shown in FIG. 5, after the patient has been positioned and prepared and the area draped, the physician wearing sterile gloves on his or her hands 22, 24, positions a sterile imaging probe or ultrasound transducer probe 20 to view the area to be anesthetized. However, it can be appreciated that the physician can use opposite hands depending if they are right or left handed or how ambidextrous they are. Particularly, the ultrasound transducer probe 20 visualizes ultrasonically the nerve bundle to be anesthetized. A local anesthetic is administered to the patient's skin and deep tissue (not shown).

As shown in FIG. 5, with hand 22 holding the ultrasound transducer probe 20, the nerve bundle to be anesthetized is identified and the needle and catheter assembly 10 is simultaneously advanced with the hand 24 into the desired position in the patient, with the aid of visualization by the ultrasound imaging. The position of the catheter 15 in relation to the position of the needle 11 is not altered as assembly 10 is inserted into the patient, due to the relative snug fit of needle 11 and catheter 15. As depicted in FIG. 6, the needle and catheter assembly 10 is advanced with the aid of visualization of the ultrasound, to the desired location, so that the tip 12 of the needle 11 is located adjacent to the nerves to be anesthetized. Once the placement of the position of the tip of the needle is accurate, the assembly is held in place by hand 24.

As shown in FIG. 7, while the needle is echogenic, it can be difficult to visualize with the aid of ultrasound and to ensure the proper placement of the tip of the needle 11, a liquid anesthetic is injected into the fluid port hub 14 of the needle, via a medical syringe 36 in hand 22. The hub 14 of the needle is in fluid communication with the opening 16 of the needle, and the liquid anesthetic is injected into the hub 14, flows through the lumen 18 of the needle and exits the needle 11, into the patient via the side opening 16, as shown in FIG. 8. The translucent property of the liquid anesthetic fluid 32 in comparison to the other structures visualized, makes it echogenic and enables the liquid anesthetic fluid 32 to be viewed by the ultrasound, since the side opening 16 in the needle 11 is angled slightly forward, so the liquid anesthetic flows out from the needle in a forward or distal direction.

As shown in FIG. 8, the echogenic liquid anesthetic 32 is observed with the ultrasound imaging and aids in identifying the tip of the needle and catheter relative to the nerve bundle. More particularly, the position of the distal end 17 of the catheter 15 can be located relative to the nerves 30, since the distal end 17 of the catheter 15 is located generally adjacent to the side opening 16 of the needle 11. This step also assures that the nerves to be anesthetized are being bathed by the fluid as they are also viewed ultrasonically.

As shown in FIG. 9, once the tip 17 of the catheter 15 and needle 11 have been properly located and are placed at the desired location in the patient, the ultrasound probe is removed. The catheter 15 is held in place with hand 24, preferably by grasping the catheter and anchoring it against the skin with two fingers, while the needle 11 is slidably withdrawn from the catheter 15 by hand 22 without dislodgement or movement of the position of the catheter. More particularly, the needle 11 is removable from the catheter 15 by holding the catheter 15 in place and slowly pulling on the fluid port hub 14 of the needle 11. As mentioned above, the needle and catheter are configured and dimensioned or sized relative to one another such that, on the one hand, the needle 11 is telescopically received within the catheter 15 to form a relatively snug fit so that the needle and catheter are moved together in a relatively fixed tandem in the patient, but on the other hand, the fit is loose enough to permit the needle to be relatively easily removed from the catheter without dislodging or altering the position of the catheter from the desired location in the patient.

Next, as illustrated in FIG. 10, in an optional step the proper position of the tip 17 of the catheter 15 can be checked. The exact placement of the tip of the catheter can be checked based on the previous verification of the location of the opening 16 of the needle 11. Therefore, it is an optional step to visualize the distal end 17 of the catheter 15. Nevertheless, to assure proper placement of the catheter 15, it is held in place by hand 24 and the anesthetic fluid is injected into the catheter 15 via the fluid port hub 13. Since the needle 11 has been removed, the fluid port hub 13 of the catheter is in fluid communication with the distal end 17 of the catheter via the lumen, and the liquid anesthetic flows into the fluid port hub 13, through the lumen of the catheter and exits the catheter into the patient via the distal end 17, to anesthetize the desired nerves. The ultrasound transducer probe 20 is optionally used once again to visualize whether the catheter is correctly positioned. Particularly, if verification of the catheter location is desired, the translucent property of the echogenic liquid anesthetic is visualized by the ultrasound imaging and can identify the position of the tip of the catheter relative to the nerves to be anesthetized. If the catheter is not correctly positioned, it is removed (not shown) and the procedure is repeated with the needle inside the catheter.

Once the desired position of the catheter has been determined to be proper, the catheter is secured to the skin with an appropriate dressing 38 and fluid port hub 13 of the catheter is attached to the desired source of anesthetic fluid via tube 34, as shown in FIG. 11. The source of anesthetic fluid can be a medication pump.

Alternatively, certain needles can be utilized which have a side tube or port intermediate of the hub, which enables a practitioner to lay the attached syringe on a sterile field.

Additionally, as an alternative method for locating the nerve bundle rather than injecting anesthetic fluid, a stimulating catheter and stimulating needle can be used which are attached to a nerve stimulator to locate the nerves. In this procedure, no liquid anesthetic is injected for visualization of the nerves or the tip of the catheter or needle as anesthetic would block the nerve conduction and the stimulator would be futile. Particularly, when using a stimulating needle with an overlying catheter, they are advanced together with the use of ultrasound through ultrasonic visualization of the tips. The catheter and needle assembly are placed near the nerve bundle by direct stimulation of the nerve which conducts impulses to the nerve causing it to twitch. Particularly, the nerve bundle is located by having the needle attached to a nerve stimulator where the voltage is dialed down to the lowest possible point while still having evidence of nerve conduction by visualizing twitches. As a result, the corresponding nerve twitches at the lowest possible amperage confirming that the needle is located close to the nerve bundle. Thus, with the aid of the ultrasound imaging, the stimulating needle is advanced until the physician sees it near a nerve bundle and the nerve twitches.

If a stimulating catheter technique for final verification is used, the stimulator is removed from the needle and placed on the catheter to verify proper placement. The simulator is activated to make sure that the nerve still twitches at low amperage. The simulator is then removed and the needle is removed from the catheter. The simulator is then re-attached to make sure that it is still near the nerve bundle and a liquid anesthetic is injected under visualization of ultrasound.

There have been described and illustrated herein exemplary method steps and apparatus for administering an anesthetic in the region of peripheral nerves. It will therefore be appreciated by those skilled in the art that yet other modifications could be made to the provided invention without deviating from its spirit and scope as claimed.

What is claimed is:

1. An assembly for continuous administration of regional liquid anesthetic, comprising:
a needle having an open proximal end, a distal end opposite therefrom, an opening generally adjacent to said distal end, and a lumen extending through said needle such that said open proximal end of said needle is in fluid communication with said opening; and
a catheter having an open proximal end, an open distal end opposite therefrom and a lumen extending through said catheter such that said proximal end of said catheter is in fluid communication with said distal end of said catheter;
wherein said needle, in a first position, is removably mounted telescopically within said catheter and is arranged to extend through said first fluid port hub and said lumen of said catheter, so that said catheter fits over said needle, and with said opening of said needle located outwardly and distally of said open distal end of said catheter and in which position said needle and catheter distal ends are advanced in tandem to a desired position within the patient and, in a second position, said needle is slidably withdrawn and slidably removed from said catheter upon hand manipulation without altering the location of said catheter within the patient.

2. The assembly according to claim 1, wherein:
said opening is located on a side of said needle.

3. The assembly according to claim 1, wherein:
said distal end of said needle is closed.

4. The assembly according to claim 1, wherein:
said distal end of said needle is blunt.

5. The assembly according to claim 1, wherein:
said catheter is relatively rigid.

6. The assembly according to claim 1, wherein:
said needle is removable from said catheter by holding said catheter and pulling on said needle.

7. The assembly according to claim 1, further comprising:
a first fluid port hub coupled to said proximal end of said catheter and in fluid communication with said lumen of said catheter; and,
a second fluid port hub coupled to said proximal end of said needle and in fluid communication with said lumen of said needle;
wherein said first and second fluid port hubs are adapted to be coupled to a source of liquid anesthetic.

8. The assembly according to claim 7, wherein:
said first and second fluid port hubs are configured and dimensioned to accept a medical syringe.

9. The assembly according to claim 1, wherein:
said distal end of said catheter is located generally adjacent to said opening of said needle.

10. The assembly according to claim 1, wherein:
said opening of said needle is angled distally.

11. The assembly according to claim 1, wherein:
said needle is a stimulating needle.

12. The assembly according to claim 1, wherein:
said catheter is a stimulating catheter.

13. The assembly according to claim 7, wherein:
said second fluid port hub is configured and dimensioned for receipt within said first fluid port hub, in a friction-fit manner.

14. A method of placing a catheter for continuous administration of a peripheral regional anesthetic in a patient, said method comprising the steps of:
providing a needle having an open proximal end, a distal end opposite therefrom, an opening generally adjacent to said distal end, and a lumen extending through said needle such that said open proximal end of said needle is in fluid communication with said opening; and a catheter having an open proximal end, an open distal end opposite therefrom, and a lumen extending through said catheter such that said proximal end of said catheter is in fluid communication with said distal end of said catheter; wherein said needle, in a first position, is removably mounted telescopically within said catheter and is arranged to extend through said first fluid port hub and said lumen of said catheter, so that said catheter fits over said needle, and with said opening of said needle located outwardly and distally of said open distal end of said catheter and in which position said needle and catheter distal ends are advanced in tandem to a desired position with the patient and, in a second position, said needle is withdrawn and slidably removed from said catheter upon hand manipulation without altering the location of said catheter within the patient;
inserting said needle and catheter assembly into a patient, towards a region to be anesthetized;
locating said distal opening of said needle with the aid of imaging; and
removing said needle from inside said catheter while holding said catheter in place in a patient.

15. The method according to claim 14, further comprising:
injecting liquid anesthetic through said needle while imaging.

16. The method according to claim 14, further comprising:
providing a first fluid port hub coupled to said proximal end of said catheter and in fluid communication with said lumen of said catheter and a second fluid port hub coupled to said proximal end of said needle and in fluid communication with said lumen of said needle; and
coupling a source of liquid anesthetic to said hub of said catheter, after said needle is removed.

17. The method according to claim 14, further comprising:
securing the location of said catheter with a topical dressing.

18. The method according to claim 14, wherein:
the step of imaging is performed with ultrasound.

19. The method according to claim 18, wherein:
the step of imaging includes injecting an echogenic liquid into said needle.

20. The method according to claim 14, wherein:
said needle is a stimulating needle.

21. The method according to claim 14, wherein:
said catheter is a stimulating catheter.
